(19) United States
(12) Patent Application Publication
(10) Pub. No.: US 2008/0288080 A1
(43) Pub. Date: Nov. 20, 2008

(54) KNEE JOINT PROSTHESIS

(76) Inventor: Kantilal Hastimal SANCHETI, Pune (IN)

Correspondence Address: LUCAS & MERCANTI, LLP, 475 PARK AVENUE SOUTH, 15TH FLOOR, NEW YORK, NY, 10016, US

(21) Appl. No.: 12/179,917

(22) Filed: Jul. 25, 2008

Related U.S. Application Data
(63) Continuation-in-part of application No. 12/064,568, filed on Feb. 22, 2008.

(30) Foreign Application Priority Data
Aug. 24, 2005 (IN) 1005/MUM/2005
Aug. 10, 2007 (IN) 1560/MUM/2007

(51) Int. Cl. A61F 2/38

(52) U.S. Cl. 623/20.24; 623/20.27; 623/20.15; 623/20.32; 623/20.35

(57) ABSTRACT

A knee joint prosthesis is disclosed. The prosthesis has a femoral component and a tibial component. The tibial component has a tibial plateau element fitted in a tibial tray element. A hemi capstan shaped bridge member is provided between replicated condyles on the femoral component. A specially shaped post is provided between kidney shaped meniscal depression on the tibial plateau element. The bridge member and the post act together to form an additional joint for load transfer during deep flexion of the prosthesis in its operative configuration.

This application claims the priority of U.S. patent application Ser. No. 12/064,568, filed on Feb. 22, 2008, which, in turn claims the benefit of priority of International Application No. PCT/IN/2006/000307, filed on Aug. 23, 2006, which, in turn, claims the priority of Indian Application No. 1005/MUM/2005, filed on Aug. 24, 2005. This application also claims the priority of Indian Application No. 1560/MUM/2007, filed on Aug. 10, 2007.

### FIELD OF INVENTION

This invention relates to knee joint prosthesis.

Particularly the present invention relates to a knee joint prosthesis having a greater degree of knee flexion.

More particularly the invention relates to a knee joint prosthesis, which replaces the articulating surfaces of the femur and the tibia.

### DESCRIPTION OF THE PRIOR ART

### Introduction

The human knee joint (60) as seen in FIGS. 1, 2 and 3 of the accompanying drawings, serves an essential function to allow individuals to lead a normal life. It is the largest and one of the most structurally complicated joint in the human body and a major joint for locomotion. This is due to the fact that it is the site of articulation of the longest levers of the lower limb (the femur and leg bones), which are characterized by the largest range of movements made in walking. Unlike the hip joint, the knee joint lacks inherent stability by virtue of bony articulations and depends upon soft tissue for its stability. The knee joint comprises important ligament such as the anterior and posterior cruciate ligaments, medial and lateral mensci and medial and lateral collaterals. Additional stability and mobility to the knee joint is provided by surrounding soft tissues including the quadriceps mechanism, the medial and lateral hamstrings and posterior capsule including popleteal fascia.

Three bones form the knee joint proper: the lower end of the femur (61, 63, 69 and 70), the upper end of the tibia (64, 68), and the patella (79), as seen in FIG. 3. The articular surfaces of the femoral condyles (63, 69), uniting with the tibia (67), are convex in the transverse and sagittal planes and are segments of an ellipsoid. The tibial facies articularis superior articulating with the femoral condyles (63, 69) consists of two shallow facets covered with hyaline semi lunar cartilage menisci (74, 78).

Each meniscus (74,78) is a ‘C’ shaped trihedral plate bent along the edge; the thickened peripheral edge is attached to the articular capsule while the sharpened edge directed into the joint is free. The lateral meniscus is more curved than the medial meniscus. The menisci serve as shock absorbers and facilitate rotary movement at the knee joint. In addition, they decrease the shallowness of the tibial plateau. The proximal tibiofibular joint (76) performs three functions: dissipation of torsional stresses applied at the ankle; dissipation of the lateral tibial bending movements and tensile weight bearing.

The articular capsule is attached at some distance from the edges of the femoral, tibial and patellar articular surfaces. On the femur, therefore, it stretches in front upwards, by passing the facies patellaris. On the sides it passes between the condyles and epicondyles with the latter left outside the capsule for attachment of muscles and ligaments, and at the back it descends to the edges of the condylar articular surface. On the tibia (67) the capsule is attached to the edges of the articular surfaces of the condyles. On the patella, it is attached to the edges of the cartilaginous surface, and as a result seems to be inserted into a ‘frame’ formed by the anterior part of the capsule. The medial and lateral ligaments originate from the medial and lateral epicondyles of the femur stretch on the sides of the joint perpendicular to their frontal axis: the ligamentum collaterale tibial (73) stretches on the medial side from the medial epicondyle of the femur (70) to the edge of the tibia and fuses with the capsule and the medial meniscus; ligamentum collaterale fibular (77) passes on the lateral side between the lateral epicondyle (61) and the fibular head (65). The fibular collateral ligament (77) is not attached to the articular capsule but is separated from it by a pad of fat. On the posterior aspect of the knee joint capsule are two ligaments merging with its posterior wall, the arcuate ligament (84) of the knee and the oblique ligament (86) of the knee.

The collateral ligaments 73 and 77 seen in FIG. 2 impart mediolateral stability to the knee joint and prevent excess varus and valgus openings.

The quadriceps mechanism includes the tendon of the quadriceps muscles of the thigh which is on the anterior aspect of the knee joint. It encloses the patella (79) as a sesamoid bone and is then continuous with a thick and strong patellar ligament (83) as seen in FIG. 3, which passes downwards from the apex of the patella (79) and is attached to the tuberosity of the tibia. From the quadriceps mechanism medial and lateral retinacular expansions open out which give additional stability. The knee joint also has two intra-articular ligaments called cruciate ligaments (72,75). The anterior and posterior cruciate ligaments connect the intercondylar eminence of the tibia to the medial surface of the lateral condyle and the posterior portion of the intercondylar eminence to the lateral surface of the medial femoral condyle respectively. These ligaments, impart stability against anterior and posterior translation of tibia over femur and also provide stability during knee flexion.

Lateral and medial hamstring muscles 80 and 82 provide medial and lateral stability. In addition they assist in the knee bending functions.

Two types of movement occur at the knee joint: (i) flexion and extension and (ii) rotation. Flexion and extension take place on the frontal axis passing through the femoral condyles. The flexion movement is polycentric, that is, about different centers, which are not fixed in one position but lie in a somewhat spiral or polycentric pathway.

During flexion, the femoral condyle and the tibial condyle rotate and glide relative to one another, with the center of rotation (centrode) of the joint moving posteriorly over the condyles of the femur with increasing flexion giving a ‘J’ curve. The range of flexion is considerable and is possible to an angle of even 140 degrees. Extension occurs until the femur and tibia are aligned. Further movement (hyperextension) is not possible because the condyles of the femur abut against the tibial condyles.

During extension, the tibia and femur follow the reverse path, with the center of rotation now moving anteriorly as the joint is extended. As a result the menisci are compressed, the collateral ligaments (73, 77) and cruciate ligaments (72, 75) strongly tightened, and the leg and the thigh locked in a single structure. In flexion the menisci straighten out, while the collateral ligaments relax because the points of their attachment come closer to each other; as a consequence, rotation on the longitudinal axis becomes possible when the knee is flexed. The cruciate ligaments (72, 75) restrict medial rotation of the leg, but, on the contrary, relax in lateral rotation, in which instance movement is limited by the lateral ligaments. In rotation, the greatest range of movements takes place in the region of the lateral condyle because the collateral fibular ligament (77), which does not merge with the articular capsule, relaxes more than the collateral tibial ligament (73). During rotation the menisci glide on the articular surface of the tibia. In addition to the indicated role of the cruciate ligaments (72, 75) in rotational movements, they also affect flexion and extension by holding the bones in a definite position and at the same time limiting movement. The structure and arrangement of the ligaments of the knee joint facilitate the maintenance of an upright position for a long period of time.

While the knee generally serves its purpose very well, various disorders of the knee cause a great deal of pain and loss of mobility and function to those who are affected with such disorder. Some knee disorders are congenital. Other disorders of the knee are brought on by bacterial infections, which may occur at any age. Disorders can also result from sports injuries or accidents, contracted diseases, or more commonly due to “wear and tear”. Perhaps the most wide spread disorder of the knee is arthritis. The term “arthritis” is generally used as a common name for the effects of several knee disorders, such as by way of example traumatic arthritis, infectious arthritis, osteoarthritis, and rheumatoid arthritis. Arthritis affecting the knee often causes such pain and discomfort that older patients cannot maintain an independent lifestyle. Each particular infirmity can affect the knee joint in a different manner. For example, malformation of joint surfaces can cause degeneration of joint, instability, deterioration of internal bone structures resulting in joint instability. Erosion of menisci can predispose to early arthritis.

The treatment for knee usually depends on the type of injury the patient has. For injuries like mild sprains, strains, and overuse, resting the knee may be one of the first treatments doctor recommends.

Treatment for serious knee joint pain requires a combination of therapies, including drug therapies, a regimen of rest and exercise, physical therapy and hot and/or cold fomentation. Aspirin, nonsteroidal anti-inflammatory drugs (NSAIDs) and corticosteroids are common medications for the treatment of many types of arthritis.

During treatment of diseased and damaged knee joints, surgery is often necessary to attempt to repair the knee. The term “knee prosthesis” applies to the artificial joint systems intended to replace the natural joint constituted by the conformation of the bottom epiphysis of the femur, by the conformation of the complementary top epiphysis of the tibia, and also by the femoro-patellar element. One of the most common procedures used in treatment of knee disorders is known as “arthroplasty” and entails the implantation of an artificial joint component into the knee. Arthroplasty has been one of the major areas of advancement in knee surgery during the past quarter century.

Prior art prosthetic knee joints have taken many different forms, depending upon the preferences of the orthopedic surgeon, the condition of the natural knee and the health, age and mobility of the patient. Knee joint prostheses, which have been generally available for a number of years, can be classified into two types. The first type is referred to as “stabilized” prostheses in which hinge and ball and socket type joints are used as substitutes for the anatomical knee joint. In this type of joint, movement of the knee is controlled and constrained by the hinge pin or ball and socket, and little reliance is placed on the surrounding soft tissues (i.e., tendons and ligaments) to stabilize the joint. These joints allow little, if any, anterior-posterior translation, lateral angulation, or rotation, as does the anatomical knee joint.

For this reason, such joints are considered to be undesirable, and may be prone to premature failure. The knee prostheses of the first type also possess significant disadvantages in that they generally involve the removal of natural ligaments and only permit motion about a single axis as opposed to the controlled rotation and translation characteristic of a natural, healthy knee.

The other type of knee joint prosthesis is generally referred to as a “condylar surface” prosthesis. In this type of joint, the respective bearing surfaces on the femur and tibia are replaced by similarly shaped and positioned prosthetic bearing surfaces, which are separate from and not directly connected to each other. This type of joint relies upon the surrounding tendons and ligaments to hold the joint together and to impart stability to the joint during movements.

This invention relates to prosthesis of the second type.

FIG. 4 of the prior art of the accompanying drawings illustrates the conventional knee joint prosthesis of the second type, which typically comprises a femoral component (33) and a tibial component (34). The femoral component (33) and the tibial component (34) are designed to be surgically attached to the distal end of the femur and the proximal end of the tibia, respectively. The femoral component (33) comprises a spaced pair of operatively downwardly convex bearing portion members (17) adapted for mutual articulation with mating bearing portion members (19) of the tibial component (34). The tibial component (34) comprises a spaced pair of operatively upwardly concave bearing portion members (19) adapted to receive the femoral bearing portion members (17), and a second intercondylar guiding portion member (15) disposed between and joining the two bearing portion members (19). Typically the tibial component (34) is adapted to be secured to the upper extremity of the resected tibia. It is provided with an operatively downward projecting stem (23) (keel) adapted to be received for cement fixation in a corresponding opening made by the surgeon in the upper part of the tibia.

FIG. 5 again illustrating prior art component depicts a first intercondylar guiding portion (14) disposed between and joining the two bearing portion members (17) of the femoral component (33), a bridging portion member (11) joining the anterior ends of the two bearing portion members (17) and a guiding portion member (14), a patellar support member (29) extending above the bridging portion member (11). The femoral component (33) is adapted to be secured to the condyles of the resected femur. The tapered pin members (20) projecting upwardly from the inner faces of bearing portion members (17) are received within corresponding openings drilled into the femur. The pin members (20) are fixed to the femur by means of cement such as poly methylmetha acrylate [PMMA]. Additionally, component (33) is provided with recesses (24) on the inner surfaces of bearing portion members (17) and patellar support member (29).

FIG. 6 illustrates the femoral bearing portions (17) of the femoral component (33), which exhibit a shape in sagittal planes similar to that of the natural femoral condyles, with the posterior part of said shape being an arc of a circle.

Motion of a natural knee is kinematically complex. During a relatively broad range of flexion and extension, the articular surfaces of a natural knee experience rotation, medial and lateral angulation, translation in the sagittal plane, rollback and sliding. The knee joint prostheses, in combination with the ligaments and muscles, attempt to allow natural knee motion, as well as absorb and control forces generated during the range of flexion. Depending on the degree of damage or deterioration of the knee tendons and ligaments, it is necessary for a knee joint prosthesis to limit one or more of these motions in order to provide adequate stability.

Various knee joint prostheses known in the prior art are summarized as under:

U.S. Pat. No. 3,795,922 discloses a ball-and-socket prosthesis having engaging locking members disposed between the femoral and tibial components.

U.S. Pat. No. 3,837,009 discloses a post that extends up from the tibial component into a slot in the femoral component and a pin or axle that is affixed to the femoral component and passes through a hole of carefully designed shape and size in the post.

U.S. Pat. No. 3,840,905 discloses knee joint wherein the femoral and the tibial components possess approximately saddle shapes, with the two components contacting one another in a substantially load-bearing intercondylar portion.

U.S. Pat. No. 4,209,861 discloses a novel knee prosthesis comprising a femoral component and a tibial component adapted respectively to be secured to the adjacent ends of the femur and the tibia, with each component comprising a spaced pair of bearing portions for articulation of the knee in the sagittal plane.

U.S. Pat. No. 4,213,209 discloses a knee joint prosthesis comprising a femoral component having laterally spaced-apart condylar portions shaped to match generally the shapes of the condylar surfaces of the femur and a tibial component having a plate-like platform portion which includes laterally spaced-apart concavities in the external surface, each of which receives and supports one of the condylar portions of the femoral component.

U.S. Pat. No. 4,892,547 discloses a partially stabilized knee joint prosthesis including a femoral component and a tibial component. The femoral component has spaced-apart condylar bearing portions, anterior and posterior intercondylar portions, and an intercondylar opening defined by edges of the condylar bearing portions and the anterior and posterior intercondylar portions. The tibial component has bearing surfaces for supporting the condylar being portions of the femoral component, and a relatively low intercondylar eminence between the bearing surfaces.

U.S. Pat. No. 5,011,496 discloses a prosthetic knee joint having an extended position, an intermediate position, and a flexed position. The motion of the joint includes a minor segment from the extended position to the intermediate position, and a major segment from the intermediate position to the flexed position.

U.S. Pat. No. 5,207,711 discloses a knee joint prosthesis including tibial and femoral components and a bearing insert designed for unicompartmental prosthetic total knee replacement and can be implanted using arthroscopic surgical techniques.

U.S. Pat. No. 5,702,458 discloses a knee joint prosthesis comprising femoral and tibial components. The femoral component includes a pair of condyles each curved generally to match the shape of an anatomical femoral condyle.

U.S. Pat. No. 6,013,103 discloses a medial pivot knee prosthesis having condylar bearing surfaces which bear upon depressions in a tibial component.

U.S. Pat. No. 6,203,576 discloses a complete knee joint prosthesis having prosthetic condyles as a part of the femoral element, wherein the prosthetic condyles have a curvature in the shape of a circular arc in their rear part, and the femoral element has, between theses prosthetic condyles, a convex cylindrical wall with an axis that concided with the axis of the circle in which the rear parts of the prosthetic condyles lie.

U.S. Pat. No. 6,264,697 discloses a condylar total knee replacement prosthesis having interacting guide surfaces for control of anterior-posterior displacement.

U.S. Pat. No. 6,699,191 discloses a knee prosthesis for the lower limb including a femur prosthetic element having a block presenting a lug running into the trochlea and adjacent to a notch from which a convex bearing surface extends, and a tibia prosthetic element having an insert with a sagittally oriented elevation defining a projection for antero-postereo stabilization.

U.S. Pat. No. 6,783,550 discloses a knee joint prosthesis, comprising a femoral component and a tibial component. The femoral component having a first portion adapted for fixable attachment to a distal end of a femur and a second portion formed with a bearing surface. The femoral component is sized so as to permit attachment to the femur of a patient without severing at least one the cruciate ligaments. The tibial component has a first surface that is adapted to cooperate with a patient's tibia, while a second surface of the tibial component is adapted to cooperate with the femoral component.

U.S. Pat. No. 6,783,551 discloses a method and apparatus for enabling access to an intramedullary canal of a femur through a femoral knee joint prosthesis which includes a first condylar portion and a second condylar portion.

U.S. Pat. No. 6,902,582 discloses an artificial joint suitable for use as an endoprosthesis for a human knee joint, having a first joint compartment formed by a first condyle and a first socket and a second joint compartment formed by a second condyle and a second socket.

U.S. Pat. No. 6,916,340 discloses a nonmodular tibial prosthesis having a retainer for a modular bearing on the superior surface of a tibial base, a nonmodular primary bearing directly molded to the base, and a mechanical release member mounted on the tibial base in contact with the nonmodular primary bearing.

U.S. Pat. No. 6,926,738 discloses a prosthesis having a tibial component and a mensical component having a rotating pin mounted within a bore of the tibial component. The meniscal component rotates on the tibial component.

With the indigenous existing conventional knee prosthesis it is not possible to bend the knee joint beyond 90°. Flexion beyond 90 degrees may cause the patient lot of pain and trauma and may even result in the slipping of the femoral component from the tibial component also the prior art prosthesis are not suitable particularly for activities such as sitting with legs crossed, or squatting.

Also during the movement of the knee joint, the femoral and the tibial components repeatedly exert great forces on the intermediate plate, which are applied in an unbalanced manner to a greater to lesser degree. In the long term, this results the imbalance of the knee joint and the abnormal stresses of the ligaments, and which may lead to loosening of the prosthesis.

Another disadvantage associated with conventional prosthetic knee assemblies is that of pinching of soft tissue located at the posterior side of the prosthetic knee assembly or impingement. Pinching of the soft tissue is likely to occur between the bearing surfaces of the femoral and tibial components when the contact points between the bearing surfaces move in the posterior direction as the flexion angle approaches the flexed position.

The relatively smaller contact surface experienced by the knee joint at high flexion angles may result in joint surface wear or cold flow of the joint surfaces. This can result in decreased bearing thickness.

A number of known knee joint prosthesis of the type that are designed to impart stability to the knee joint by mechanical action are not able to give deeper flexion.

### SUMMARY OF THE INVENTION

One object of the present invention is to provide a light weight knee joint prosthesis, which closely replicates the function of a natural knee.

Another object of the invention is to provide a knee joint prosthesis which offers a greater degree of knee flexion and rotation with improved stability.

Another object of invention is to provide a prosthetic knee joint with an ability to resist dislocation at high degrees of flexion and therefore permit flexion even beyond 90 degrees without pain or trauma with a satisfactory load transfer pattern.

Yet another object of the present invention is to reduce if not eliminate the likelihood of impingement and pinching of the soft tissue located on the posterior side of the prosthetic knee.

Still another object of this invention is to provide a knee joint prosthesis that in its operative configuration permits a patient to recover standing up and walking abilities as soon as possible post operatavily and which permits smoother natural movement over prolonged periods of time with as little pain, trauma and wear of the prosthesis and particularly bearing surfaces.

One other object of this invention is to provide a prosthesis in which optimum load transfer is achieved from the femoral component to the tibia via the tibial component elements.

Still one more object of the invention is to provide a prosthesis which requires less bone resection of the femur and the tibia and therefore results in greater bone sparing.

To achieve these and other objects there is provided in accordance with this invention a knee joint prosthesis comprising (i) a ‘U’ shaped metallic femoral component with one arm longer than the other, the longer arm having an operatively inwardly concave depression within which a patella can be accommodated, the shorter arm being contoured to replicate two femoral condyles of an anatomical knee; a recess provided between said replicated femoral condyles; (ii) a tibial component consisting of a metallic tibial tray element and a tibial plateau element of synthetic polymeric material rigidly shrunk fit in the tibial tray element; said tibial plateau element being hemi oval and having formed therein two laterally spaced apart, kidney shaped trough like meniscal depressions for receiving the said replicated femoral condyles; (iii) articulating means on the femoral and the tibial component including a hemi capstan shaped bridge member, having an operative concave surface, on the femoral component and a post, having an operative convex surface complementary to said concave surface, on the tibial component; and (iv) load transfer means comprising triangular web shaped flanges and a stem extending from the operative lower surface of the tibial component.

Typically. the femoral component and the tibial tray element are of cobalt chrome alloy and the tibial plateau element is of high density synthetic polymeric material, typically high density polyethylene.

Typically, said bridge member is disposed in said shorter arm between said replicated condyles and bridges said recess in the femoral component.

Typically, said recess in said femoral component has at least one window through at least a portion of the recess.

Typically, the end of the recess distal from the bridge member is concaved.

Typically, the longer arm of femoral component terminates in a curved edge. In accordance with one preferred embodiment of the invention, projecting pins extend operatively inwardly from the inner surface of the U shaped femoral component on either side of the recess.

Preferably, the operative outer surface of the femoral component is mirror polished and the operative inner surface defines a plurality of recesses for securing the femur to the femoral component in its operative configuration.

Typically, the post extends operatively upwards from the tibial plateau between the meniscal depressions.

In accordance with a preferred embodiment of the invention, the post is defined by a truncated pyramid rounded at the top, sectioned at the center along a right angularly disposed convex surface, said post being disposed on the operative posterior side of the tibial plateau between the meniscal depressions.

Typically, the post has an operative anterior wall which has a convex smooth surface concaved at the edge joining the post to the tibial plateau, said anterior wall contouring the hemi capstan shaped bridge member of the femoral component in its operative configuration.

In accordance with one embodiment of the invention the post has a reinforcing pin provided therein.

In accordance with a preferred embodiment of the invention the triangular web flanges on either side of the stem define walls joined to the tibial tray, said walls being operatively below and aligned with the short axis of the meniscal depressions in the tibial plateau and extend around the deepest point of the meniscal depressions approximately below the contact area between the contacting bearing surfaces of the replicated condyles and the surfaces of the meniscal depressions in the operative configuration of the prosthesis.

Typically, the stem is defined by a cylindrical body having a long axis extending operatively at an angle between 5 to 10 degrees, preferably 7 degrees to a vertical below the tibial tray.

Typically, the base of the stem joined to the base of the tibial tray lies approximately below the operative anterior edge of the base of the post whereas the free edge of the stem extends upto the posterior edge of the base of the post.

### BRIEF DESCRIPTION OF THE DRAWINGS

The invention will be described in detail with reference to a preferred embodiment thereof, which is total knee replacement. Reference to this embodiment does not limit the scope of the invention, which is limited only by the scope of the claims.

In the drawings:

FIG. 1 illustrates the posterior view of the two bones of the knee joint;

FIG. 2 illustrates the anterior view of the two bones of the knee joint;

FIG. 3 illustrates the side view of the bones of the anatomical knee joint;

FIG. 4 illustrates the back view of the artificial femoral and tibial components according to the prior art;

FIG. 5 illustrates the oblique view of the artificial femoral and tibial components;

FIG. 6 illustrates the artificial femoral and tibial components in the operative configuration;

FIG. 7 illustrates the rear view of the artificial femoral and tibial components indicating the sitting position according to the present invention;

FIG. 8 illustrates the side view of the artificial femoral and tibial components of FIG. 7,

FIG. 9 illustrates the side view of the artificial femoral and tibial components in the operative deep flexion configuration;

FIGS. 10 and 11 illustrates the front isometric and front view of the artificial tibial plateau;

FIGS. 12 and 13 illustrate the front and side view of the tibial plateau;

FIGS. 14 and 15 illustrate the sectional views of alternative posts of the tibial plateau;

FIGS. 16 and 17 illustrate the front isometric and front view of the artificial tibial tray component;

FIGS. 18A and 18B illustrates the side view of the tibial component showing the notches used to fix the tibial plateau onto the tibial tray;

FIG. 19 illustrates the side view of the artificial femoral component;

FIG. 20 illustrates the back view of the femoral component;

FIGS. 21A and 21B illustrate the bottom view of alternative embodiments of the femoral component;

FIG. 22 and FIG. 23 illustrates the top and the front view of the patellar component;

FIG. 24 illustrates the knee joint after knee joint prosthesis;

FIGS. 25 and 26 illustrates the rear view and the front view respectively of the knee joint with the knee joint prosthesis; and

FIGS. 27 and 28 illustrates the movements of the knee joint after knee joint prosthesis.

FIGS. 29 and 30 show an alternative embodiment of the invention showing a femoral component with a window for insertion of an intramedullary device.

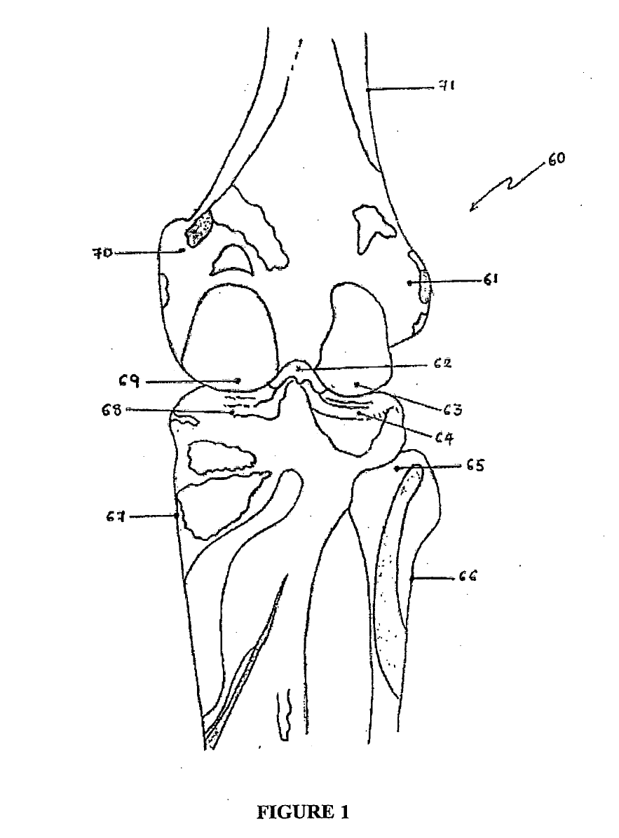

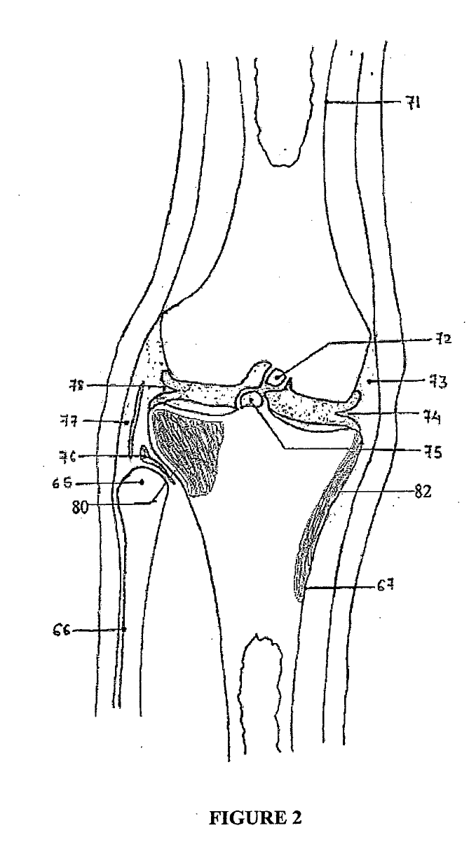

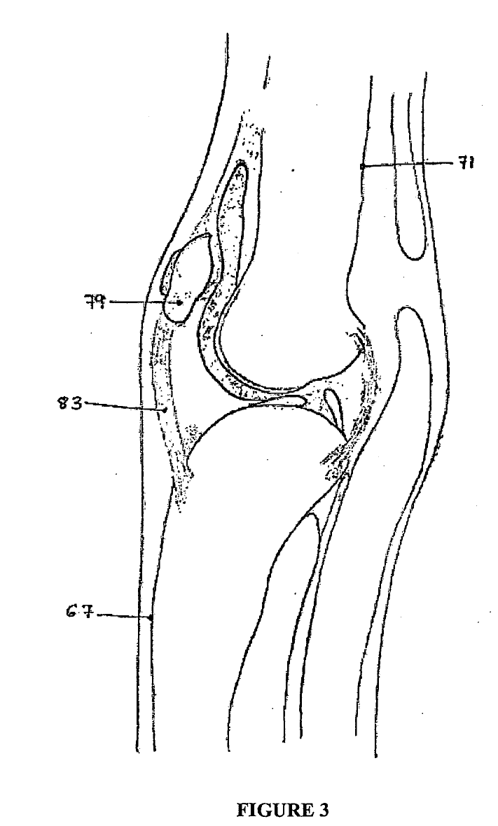

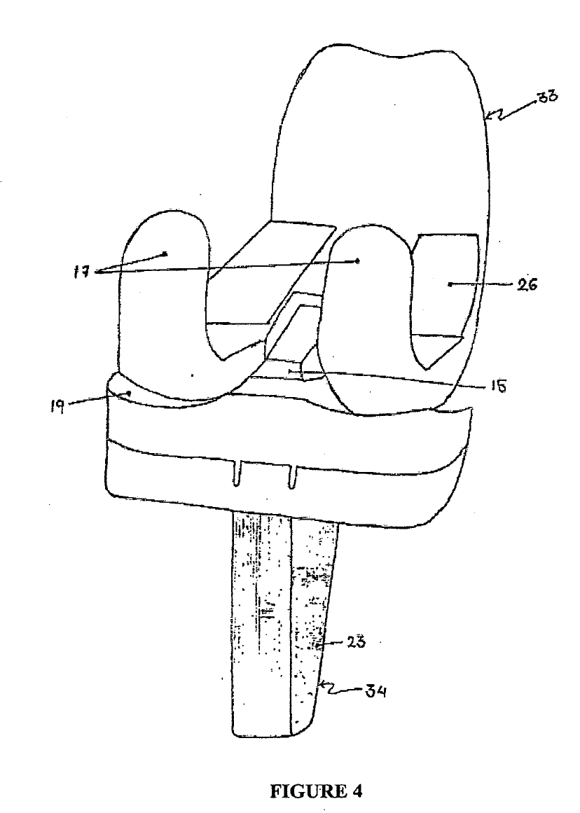

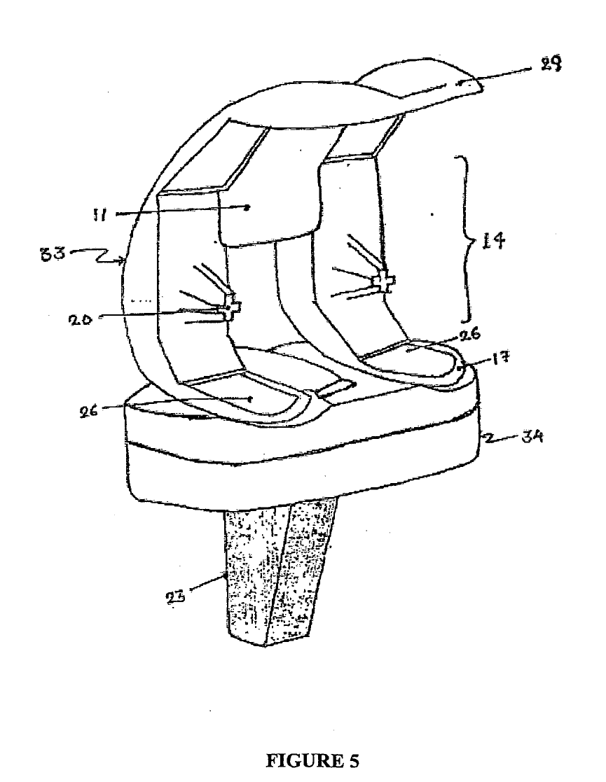

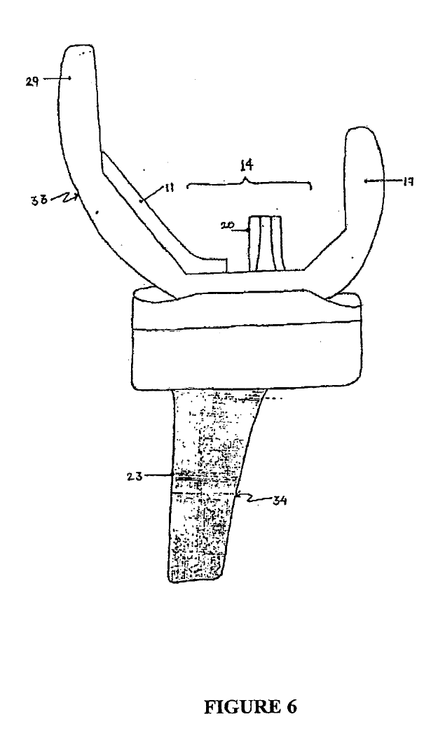

### DETAILED DESCRIPTION OF THE INVENTION

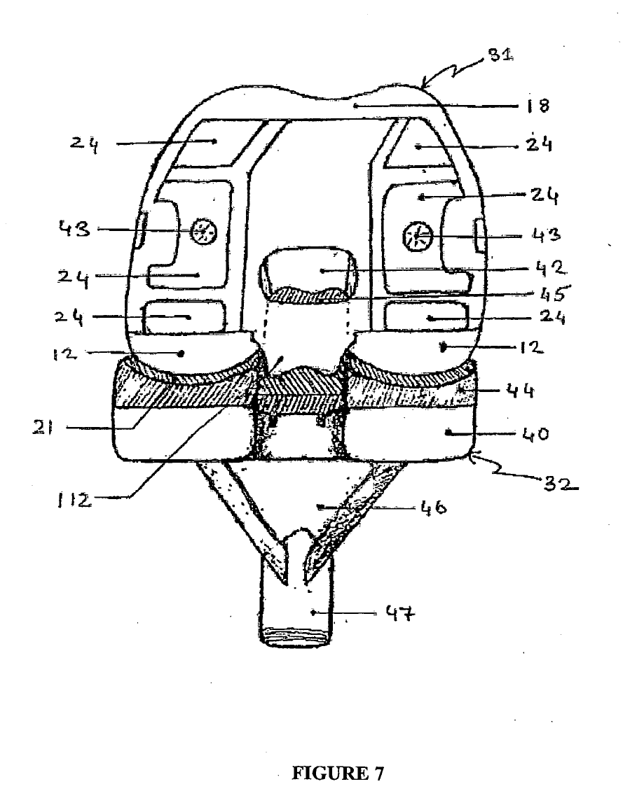

The present invention will now be explained with reference to the FIGS. 7 to 28 of the accompanying drawings which is illustrative of some preferred embodiments in accordance with this invention.

FIG. 7 illustrates the artificial femoral component (31) and the tibial component (32) according to the present invention. The femoral component (31) is designed to cooperate with the tibial component (32) in simulating the articulating motion of an anatomical knee joint: a rocking movement along a vertical axis permitting extension and flexion of the knee and an anterior posterior slide of the femoral component on the tibial component and a valgus varus rotation cum gliding of the two components along a vertical axis.

The femoral component (31) comprises a ‘U’ shaped body with one arm (18) longer than the other arm (12), the longer arm 18 has a concave depression (33) [not seen in FIG. 7] on its outer surface within which a patella (anatomical 79 or prosthetic 115 not seen in FIG. 7) can be accommodated, the shorter arm 12 is contoured to replicate two femoral condyles of an anatomical knee. A recess 16 is provided between said replicated femoral condyles 12. The replicated condyles act as a pair of operatively downwardly convex bearing members connected by a hemi capstan shaped bridge member 112. The replicates condyles are adapted for mutual articulation with meniscal depressions 21 of the tibial component (32), which is described in detail hereinbelow.

The term “capstan shaped” used in this specification is defined to mean an element which has a curved body having its narrowest portion in the middle and which has an increasing radius as it approaches the ends. The term “hemi capstan” is defined to mean substantially half of a capstan shaped body cut across body end to end.

The femoral component is provided with two cylindrical, tapering projection pins (43). Typically an intercondylar opening (42), (42a) is provided in the recess (16) of the femoral component (31). The opening may extend throughout or only through a portion of the base of the recess 16. Separate right and left femoral components are provided for the right and left knee respectively in different sizes.

The tibial component (32) comprises: a tibial plateau (44) and a tibial base or tray (40). Typically a recess (110) on the operative posterior side of the tibial component (32) reduces the overall weight of the tibial component (32). The upper portion of the tibial tray (40) has notches (111), which help in the better fixation of the tibial plateau (44) over the tibial tray (40). The tibial tray has a load transfer member which is inserted into the tibial bone. The load transfer member consists of a cylindrical projection stem (47) with triangular web shaped flanges (46) which is the part of the tibial tray (40) that actually enters the tibial bone (67).

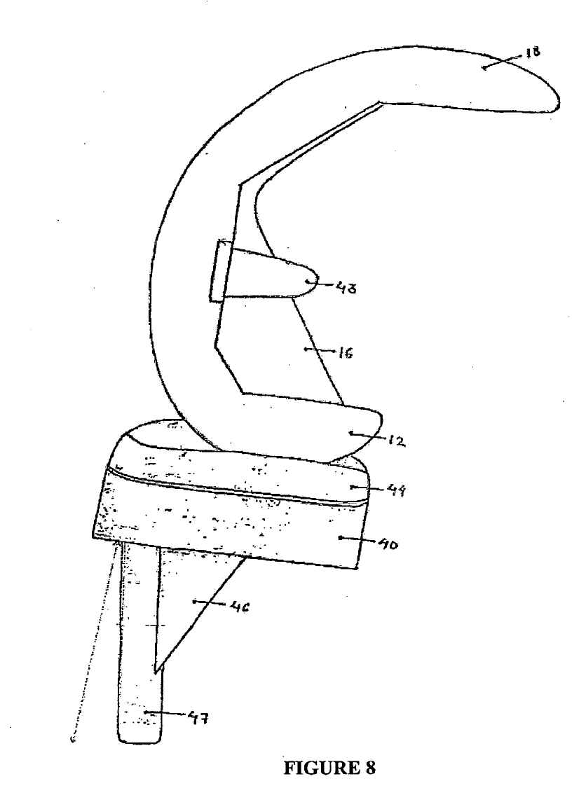

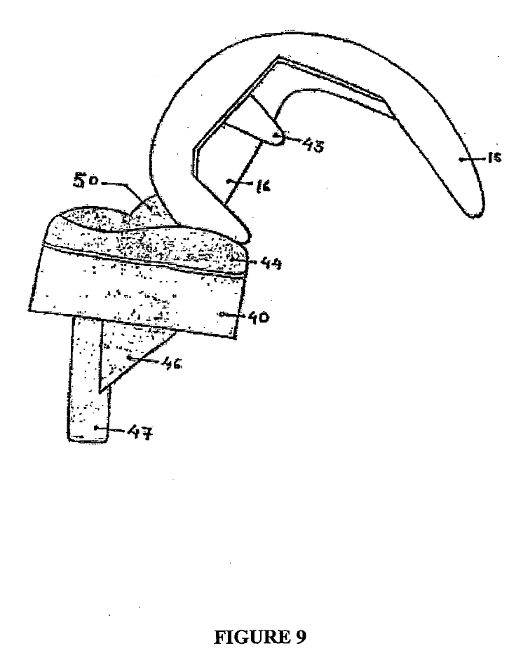

FIG. 8 and FIG. 9 illustrate the femoral and the tibial components configured in such a way that the rotation of the femoral component (31) with respect to the tibial component (32) about the longitudinal axis of the tibia is facilitated by the proximity of the contact area of femoral bearing portion members (12) upon tibial bearing portion members (13) to the longitudinal axis of the tibia, and the prosthesis is capable of accommodating about 15 degrees of varus-valgus movements without one of the femoral bearing surfaces (46) lifting above the corresponding tibial bearing surface (21). Different sizes of tibial components separately for the tibial tray and the tibial plateau are provided and elements are selected for individual patients.

The prosthetic knee joint is under compressive loading during normal activities. The Valgus-varus stability of a knee joint refers to the ability of the joint to resist the lateral forces or rotary forces that would cause rotation of the tibia relative to the femur in the frontal plane. The lateral forces or the rotary movements that cause rotation of the tibia relative to the femur in the frontal plane tend to create a dislocation. Such dislocation is particularly likely to occur on either the medial or lateral side of the prosthesis, depending upon the direction of the lateral forces. The interaction of the intercondylar guiding portions (14) and (50) provides, in addition to the desired backward guidance of the femoral component upon the tibial component (32) with flexion of the knee, a highly desirable amount of stability against undesired movements and dislocations of the artificial knee, without causing the knee joint prosthesis to be unduly restrictive, cumbersome or uncomfortable in actual use in the body of the patient. The enhanced stability will compensate for the loss of the cruciate ligaments, which must be severed during implantation of the prosthesis but have often already been rendered useless in cases of moderate deterioration of the natural knee joint such as that caused by arthritis.

### DESCRIPTION OF THE COMPONENTS OF THE PRESENTLY PREFERRED EMBODIMENTS OF THE PRESENT INVENTION

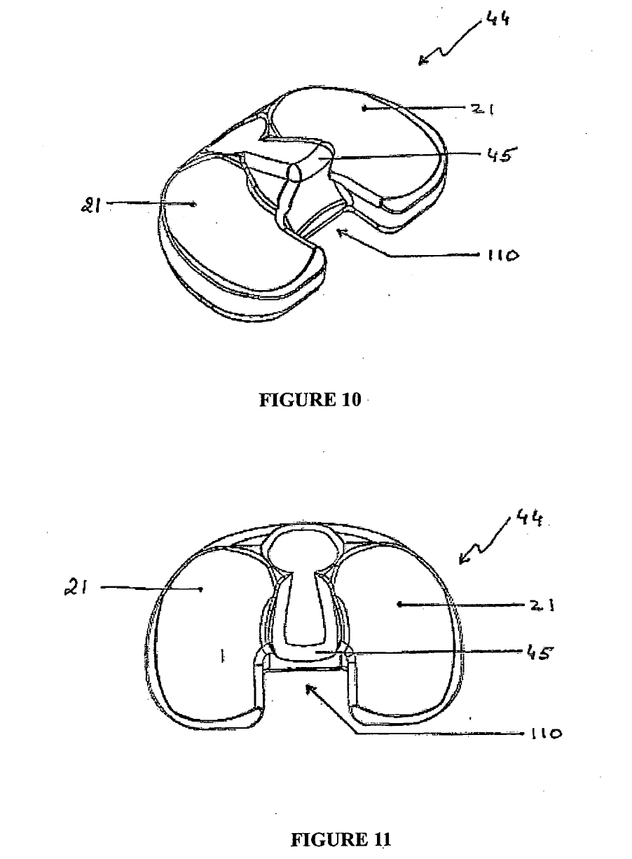

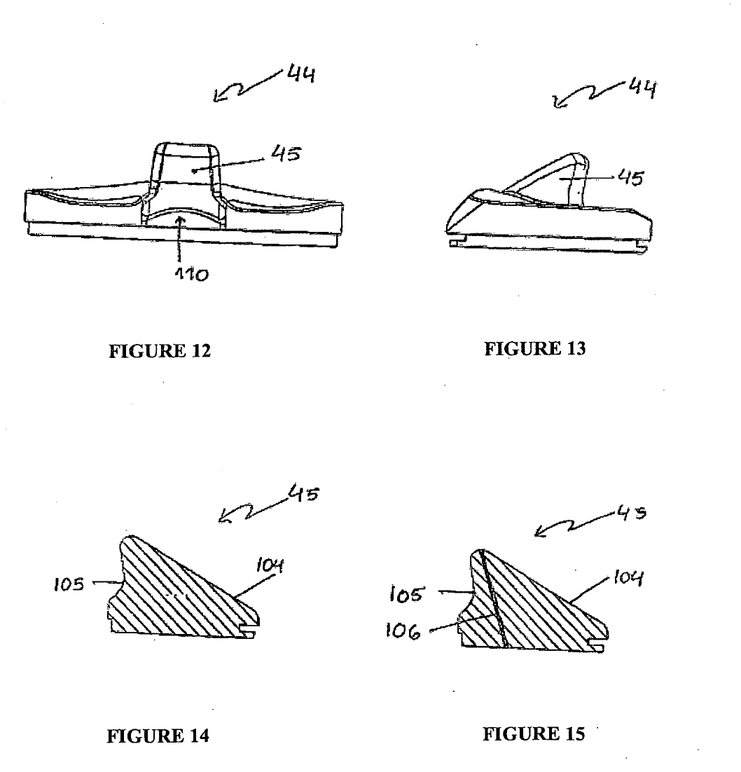

FIGS. 10 and 11 illustrates the front and side view of the artificial tibial plateau element. FIGS. 12 and 13 show the geometry of the tibial plateau. It is approximately hemi oval forming two laterally spaced apart, trough like kidney shaped depressions referred to as the meniscal depressions or condylar bearing portions (21) which are used to receive the replicated femoral condyles. The meniscal depressions (21) are separated posteriorly by a notch (110). The operative surface of the plateau slants from the anterior to the posterior edge, i.e. it is relatively higher in front and recedes in height at the rear. The raised front end limits forward slide whereas the rear profile assists in flexion of the knee beyond ninety degrees during which movement the novel hemi capstan and post engagement provides stability to the movement.

FIGS. 14 and 15 illustrates a stabilized intercondylar post (45) extending upwardly from the plateau (44) portion between the depressions. The intercondylar post (45) is defined by a truncated pyramid rounded at the top, sectioned at the center along a right angularly disposed convex surfaced posterior wall (105), and a slanting concave surfaced anterior wall (104) said post being disposed on the operative posterior side of the tibial plateau between the meniscal depressions.

The operative posterior wall 105 which has a convex smooth surface concaved at the edge joining the post to the tibial plateau, said posterior wall contours the hemi capstan shaped bridge member 112 of the femoral component in its operative configuration. The edges of all the lateral and top surfaces of the intercondylar post (45) are rounded thereby assisting in the smooth movement, particularly rotation of the femoral component (31) over the tibial plateau (44) and hence reducing wear and tear. The intercondylar post (45) functions as a hyperextension stop thus avoiding dislocation of the femoral component (31) as may occur in a conventional knee joint prosthesis. The anterior wall (104) of the intercondylar post (45) is concave at the bottom and is slanting from the apex of the post to the base where it joins the tibial plateau. The use of the post and hemi capstan shaped bridge member on the femoral component allows for rotation freedom of the prosthesis in spite of the tibial component being a monoblock unibuilt rigid component. A reinforcing pin (106) may be provided, as seen in FIG. 15 in the body of the post 45.

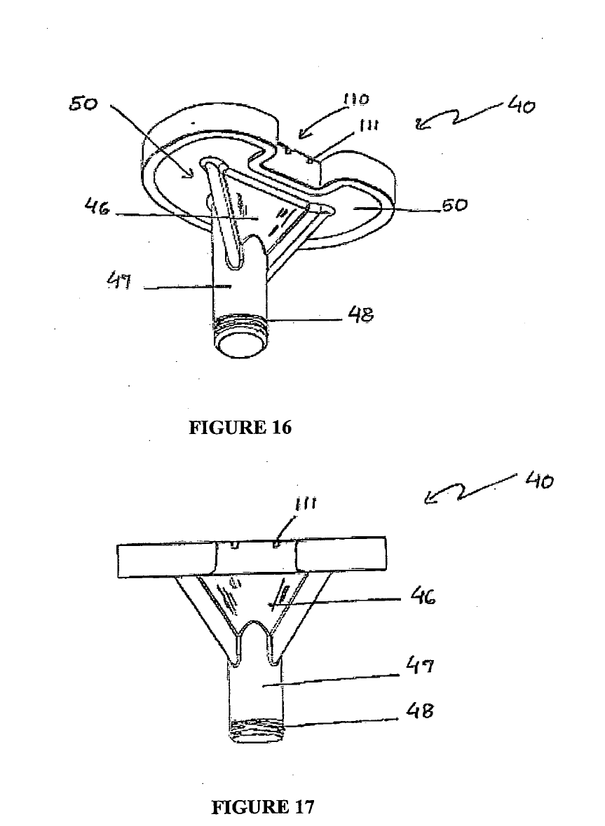

FIGS. 16 and 17 illustrate the bottom isometric view and rear view of the artificial tibial tray component. The metal tibial tray (40) is the part of the prosthesis that is fixed on the tibial bone. It is made of cobalt chrome alloy. The tibial plateau (44) is fitted on the top surface of the tray. The tibial plateau is made of high-density polyethylene and is fixed over the tibial tray by interlocking mechanism and shrink fitting. This is achieved by cooling the plateau to around −70 degrees celsius by dry ice and methanol and placing the plateau in the tray. PMMA [poly methyl meta acrylate is used as the load transferring material between the total joint prosthesis and the bone implantation site.

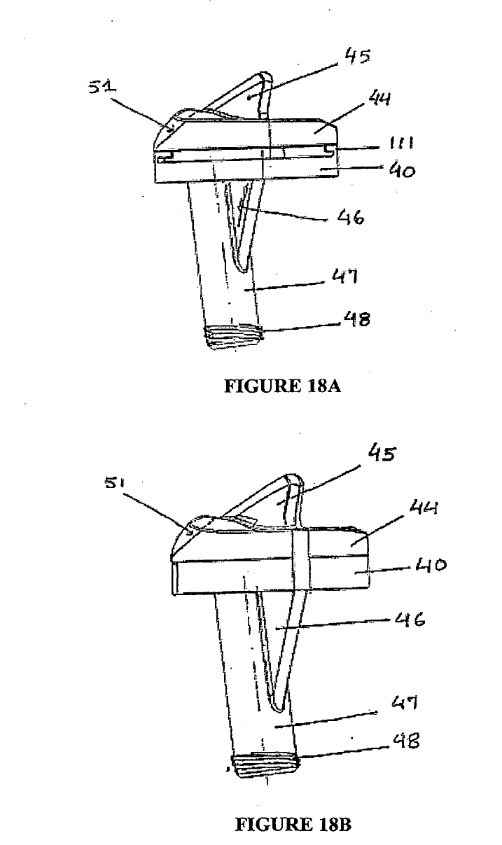

A recess (50) is provided at the bottom surface of the tray, which receives the bone cement required for bonding the chamfered tibial bone (67) and the tibial component (32). Typically a cylindrical projection stem (47) with a triangular web shaped flange (46) is part of the tibial tray that actually enters the tibial bone (67). The shape of the projection stem (47) and the flanges (46) are made such that it provides better and stronger fixation. In addition, the web flanges also give rotational stability to the prostheses of this invention. The base of the stem joined to the base of the tibial tray lies approximately below the operative anterior edge of the base of the post whereas the free edge of the stem extends upto the posterior edge of the base of the post. The walls of the flanges (46) are operatively below and aligned with the short axis of the meniscal depressions in the tibial plateau and extend around the deepest point of the meniscal depressions approximately below the contact area between the contacting bearing surfaces of the replicated femoral condyles and the surfaces of the meniscal depressions in the operative configuration of the prosthesis. This is done to ensure that the entire load of the femur is transferred to the tibia. FIGS. 18A and 18B illustrates the side views of the tibial component (32) in a slightly exploded and a fitted view showing the notches (111) used to fix the tibial plateau (44) on the tibial tray (40). A concave cusp depression (51) is formed at the front end of the tibial plateau to locate the patella and its tendons in the flexion configuration of the prosthesis.

The end of the stem distal from the tibial tray is provided with threads to accommodates extension rods for additional support for the prosthesis. Extension rods [not shown] can be screwed over the threads (48) on the stem [keel] portion of the tibial component which can be used for neuropathic joints, a tibia having severe bone loss or ligamentous insufficiency. Further the design of the tibial component requires less tibial resection as a tray of lesser thickness can be used because of the mono-block uni-built design.

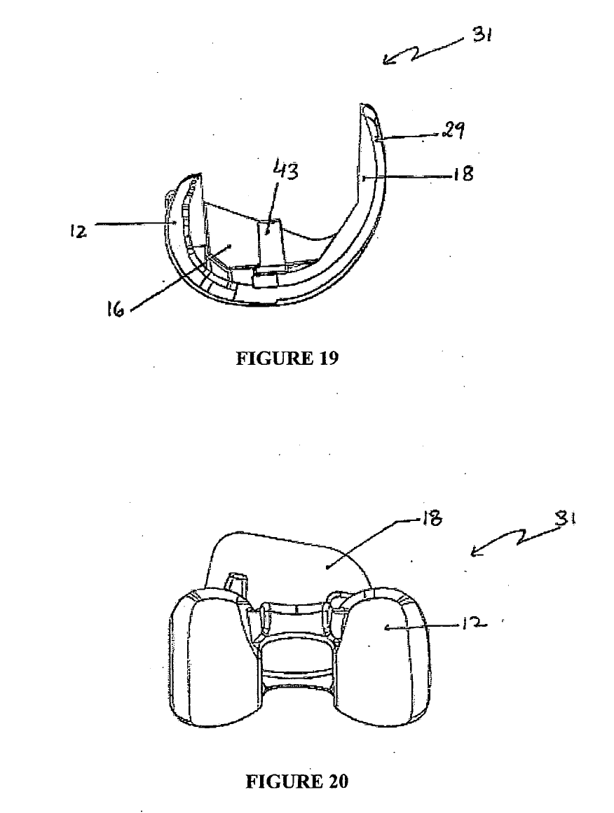

FIGS. 19, 20 and 21A and 21B illustrate the femoral component (31) which is a single piece component typically made of biocompatible high strength, durable metal, such as a cobalt chromium alloy and fixed on the femur (71) using biocompatible bone cement. The percentage composition of various elements in the cobalt chromium alloy is

### Chromium: 27 to 30%

### Molybdenum: 5%

### Carbon: 0.35%

### Iron: 1.5%

### Nickel: 1%

### Silicon: 0.4%

### Manganese; 1%

Cobalt: balance.

The femoral component is made by investment casting the molten metal by preparing a die of required shape.

The outer part of the femoral component (31) is U-shaped with one arm (18) longer than the other (12) as depicted in FIG. 19. The longer and the shorter arm of the U-shaped femoral component are inwardly curved thereby giving it a wrap-around design so that better geometric contact can be made with the end of the femur. The design of the femoral component permits lesser condylar resectioning with the same stability which results in bone sparing and less femur bone resection. A depression (29) in the longer arm (18) acts as a patellar support. The arm (18) has an inwardly concave depression (29) within which a patella can be accommodated. The shorter arm (12) of the U-shaped femoral component is contoured to replicate the femoral condyles of the anatomical knee. These curved surfaces act as condylar bearing surfaces of the femoral component. An operatively downwardly slanting recess 16 is disposed in between the condylar bearing surfaces. A hemi capstan shaped element (112) bridges this recess 16 at the posterior side of the femoral component in the shorter arm. The hemi capstan shaped element (112) is convexed with a particular predetermined radius of curvature. An oval intercondylar opening (42) or (42A) is provided on the bridging member, which helps accommodate intermedullary nails extending into the femur for better fixation in the case of multiple trauma (fractures of the femur). The end of the recess distal from the bridge member is concaved. The design of the recess in the intercondylar region and also the size of the opening also spares bone resection in this region resulting in over 20% saving of bone during the surgery.

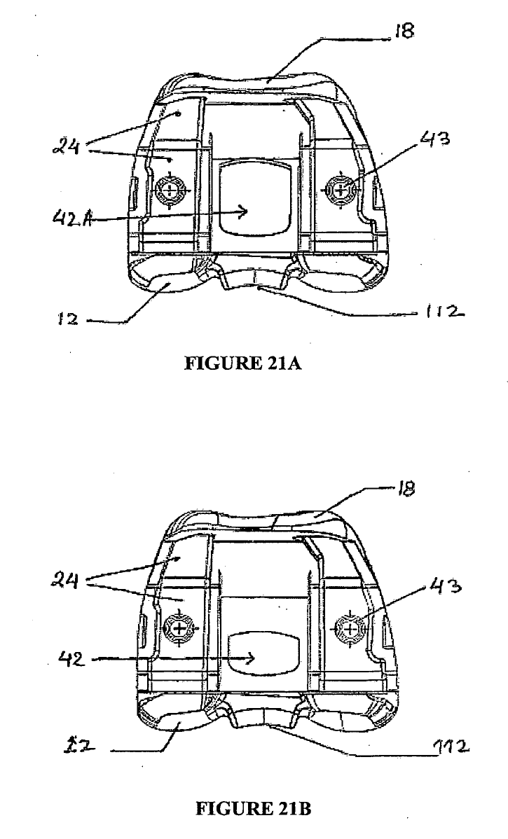

FIGS. 21A and 21B illustrate the inner part of the femoral component (31) which is precisely machined to form well-defined edges. The end of the femur (71) is chamfered and resectioned such that it matches these edges, thus enabling exact fixation of the femoral component (31) onto the chamfered resectioned femur. In one embodiment polygonally shaped recesses (24) are present on this inner part of the femoral component (31), these recesses (24) accommodate bone cement used to bond the resectioned femur to the femoral component. Typically two cylindrical, upwardly tapering projections (43) are provided on the inner part of the femoral component, which help in the better fixation of the femoral component onto the femur by bone cement.

The hemi capstan element (112) and the post (45) in the operative configuration of the prosthesis of this invention in deep flexion not only substitutes for the cruciate ligaments which necessarily need to be severed during the operative process, but act as an additional joint in addition to the replicated medial and lateral condylar joints of the femoral (31) and tibial (32) components, for transferring a portion of the load in deep flexion. This reduces the load on the condylar joints and therefore reduces the wear in the mensical depressions of the tibial plateau. Load is therefore shared between the replicated condylar joints and the joint between the hemi capstan element (112) and the post (45).

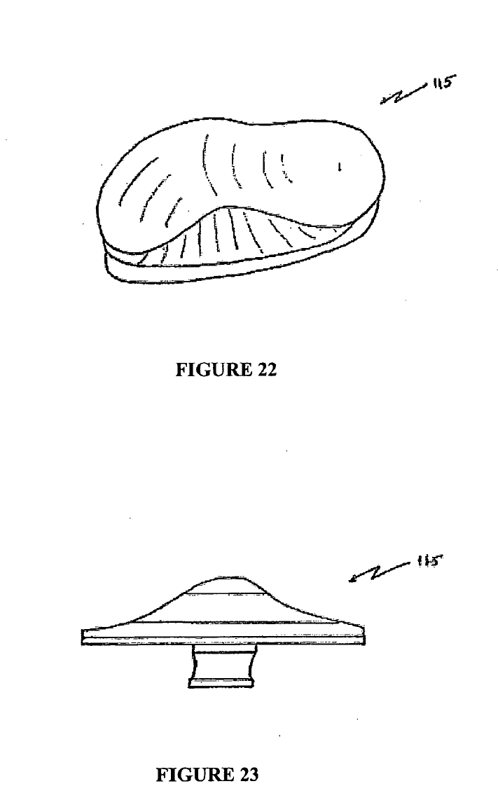

FIG. 22 and FIG. 23 illustrate the artificial patellar component. The femoral component has a large support in front for contact with the gliding patella. The patellar component (115) duplicates the shape of the natural kneecap and is typically made of polyethylene. The kneecap protects the joint, and the resurfaced patellar button slides smoothly on the front of the joint.

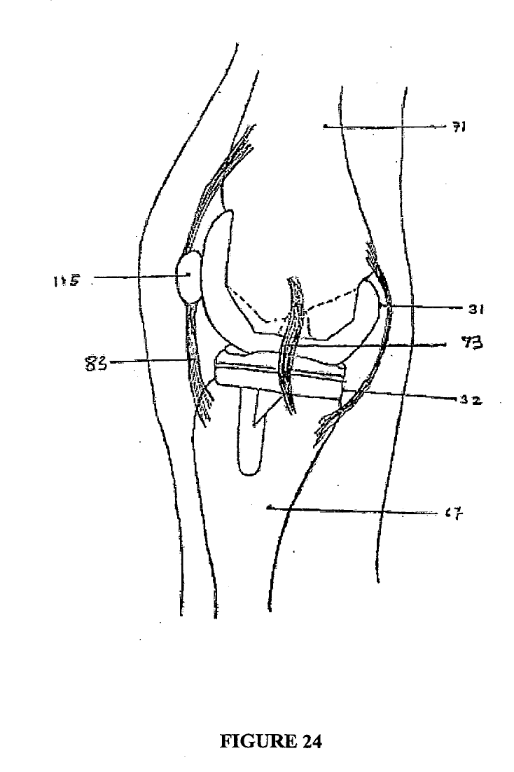

FIG. 24 illustrates the knee joint prosthesis with the artificial femoral component (31), tibial component (32) and patellar component (115). To ensure the smooth movement and to avoid the slippage of the femoral component (31) and the tibial component (32), the tibial tray (40) of the tibial component (32) is fitted typically at an angle of 7 degrees with respect to the insertion stem (47) extending into the medullary canal of the tibia. It is to be understood that the FIGS. 24 to 28 and other anatomical representation are provided for illustration purposes only and are not anatomically accurate in positioning or dimensions.

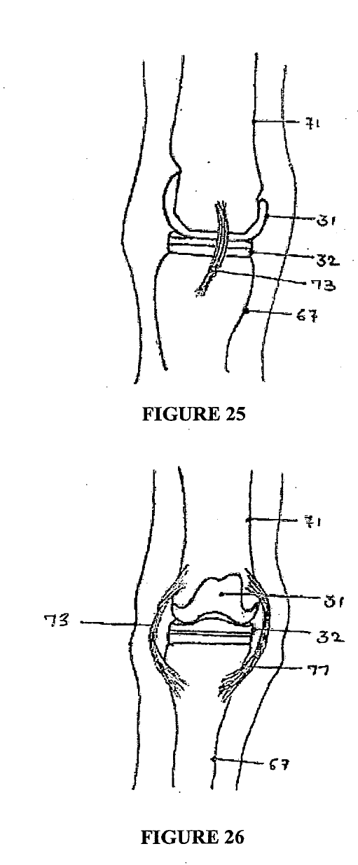

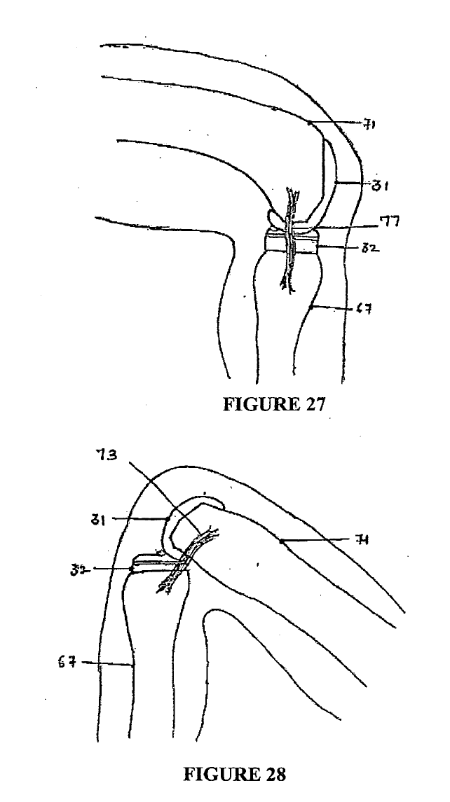

FIGS. 25 and 26 illustrate the side view and the front view respectively of the knee joint prosthesis in extension. FIGS. 27 and 28 illustrate the movements of the knee joint prosthesis in flexion and deep flexion respectively. The figures clearly show the balancing importance of the collaterals (73 and 77)

In addition to the flexion and extension movement of the femoral component on the tibial plateau of the tibial component, there is a rolling cum gliding action of the femoral component on the tibial plateau. The femoral component not only rolls on the tibial component but there is a gliding movement such that in the extended configuration of the prosthesis as seen in FIG. 24, the condylar recess abuts the anterior wall (104) of the post whereas in the flexed configuration upto around 90 degrees the femoral component glides forward until it abuts the posterior wall of the post. For further flexion beyond ninety degrees, there is no further gliding action of the femoral component and the hemi capstan element rolls and is therefore angularly displaced on the posterior wall of the post during which rolling the geometry of the meniscal depressions of the tibial plateau and the condylar surfaces of the femoral component assist in joint stability. In this configuration a portion of the load is transferred from the condylar surfaces to the hemi capstan and post elements.

The femoral component (31) and the tibial component (32) can have various other configurations, shapes, and dimensions. The various configurations can be chosen in light of the size of knee, the amount of damage to knee, the cooperation between tibial component (32) and femoral component (31), or other reasons that will be appreciated by one skilled in the art. In reconstructing the knee in accordance with the present invention, the operating procedure is as follows:
Incision of 10-12 cm is taken over the affected knee joint.
The knee joint is first exposed and the patella with attached ligaments is laid to one side
All the damaged bone and cartilage is removed.
Patellar bone is everted and prepared.
Femoral intra-medullary rod is placed and a special cutting jig is placed on the end of the femur. This jig is used to make sure that the bone is cut in the proper alignment to the leg's original angles. The jig is used to cut several pieces of bone from the distal femur so that the artificial knee can replace the worn surfaces with a metal surface.
The top of the tibia is cut using another jig that ensures the alignment is satisfactory. The cut is taken perpendicular to long axis at a distance of 8-9 mm from the healthy bone.
Marking the anatomical points for proper placement of component.
Femoral size is used with reference to the anterior reference line, posterior reference line, medial and lateral referencing line.
Makes the selection of desired implant easy.
Tibia cut surface is prepared and proper size of tibial component is selected.
If larger defects are present then in spite of using expensive wedges, reconstruction of defected part, using patient's own bone and screws is done.
Notch cut and chamfer cuts are taken with a jig
Components are fixed to bone with the help of quick setting polymethylmethacrylate (PMMA) bone cement; the knee is maintained in desired position till cement sets in.
Patella tracking is checked.
Patients knee is mobilized immediately, once surgical pain subsides.

The knee joint prosthesis, as implanted in the reconstructed knee joint, permits substantially the full function provided by the anatomical knee joint.

Having now fully described the invention, it will be apparent to one of ordinary skill in the art that many changes and modifications can be made thereto without departing from the spirit or scope of the invention as set forth herein.

### Clinical Experimentation:

### Case 1:

A 58 year old female patient presented herself with severe pain in the right knee an inability to carry activities of daily living with deformity in her right knee, Patient had undergone total knee replacement surgery for her left knee using conventional indigenous knee prosthesis. The right knee was operated for TKR using the prosthesis of this invention. The surgical procedure was performed under spinal and epidural anesthesia in supine position using tourniquet and side and distal posts. An anterior midline incision was taken. Medial capsulotomy was performed after capsular marking using a sharp scalpel. The Patella was everted and locked in the everted position and resurfaced. The femoral and tibial osteophytes were removed to obtain a better anatomical shape of the femoral and tibial condyles. Medial peritibial release was done for ligament balancing. Femoral and tibial cuts were taken followed by sizing for the prosthetic components. Medium plus femoral component and medium tibial components were selected. Trial reduction was carried out and medio lateral and antero posterior stability were assessed. Tibial keel preparation was performed using a selected tibial base plate and tibial notch cutting guide. A thorough lavage with normal saline was given using a pulse lavage machine. The bone surfaces were dried and cementing was performed first on the femur and patella and then on the tibia using PMMA bone cement. Excess of cement was removed from each component and the joint was reduced after setting of the cement. The tourniquet was released. Bleeders were identified and coagulated with thermal cautery. The Patellar tracking was assessed by doing full flexion of the knee. The joint was thoroughly lavaged and closed in layers over a drain. Drain was activated immediately after the surgery and a second activation was done after 24 hours. Drain was removed after 48 hours with a blood loss 150 cc. The patient was started with static exercises on the same day and was made to stand on the following day. Ambulation of the patient using a walker and full weight bearing was started on the third day. The patient was allowed to walk using a tripod stick in the left hand on the tenth day and was taught stair climbing on the 11^{th} post operative day. The knee range of motion exercises was given right from the second post operative day and 90° flexion was obtained on the fifth post operartive day and 110 degree on the tenth post operative day. Sutures were removed on the 13^{th} post operative day and the patient was asked to walk using stick support for further tree weeks. Patient was discharged on the 13^{th} day and a follow up examination was carried out after six weeks of surgery. The patient was able to bend her knee upto 130 degrees without pain and was able to sit cross legged on the right side without pain. At the end year the patient was having the same range of motion with ability to sit cross legged on the right side without pain or instability.

Similar procedure was carried out on 124 patients 43 male and 81 female in the age group of 30 to 90 years. In 16 patients bi lateral surgery was performed and both knee joints were replaced. 61 of these patients presented with osteo arthritis whereas 52 with rheumatoid arthritis. 2 patients were suffering from post traumatic arthritis whereas 8 had arthritis of the neuropathic variety. One patient had pigmented villo nodular arthritis. Similar procedure was performed as in case on all the patients except that in 35 of them patelloplasty was performed instead of patellar resurfacing.

On an average by the second day after surgery most patients were made to walk with a walker. By the third day they were made to stand unaided. By the 10^{th} day the patients were made to walk with a tripod walking stick. By the 11^{th} day most patients were taught to climb stairs. By the 13^{th} day most patients were discharged but were advised to walk with a stick for three weeks. As far as knee flexion is concerned most patients were able to flex the knee upto ninety degrees on the fifth day itself. This flexion increased to 110 by the 9^{th} day. After 3 weeks the flexion was 110 to 120 degrees and after 45 days in many cases this was even beyond 125 degrees without any pain or discomfort never before seen in prior art prosthesis.

The prosthesis in accordance with this invention can be applied universally to all cases where knee joint replacement is required because of the inherent stability of the tibial component.

According to another aspect of this invention there is provided a knee prosthesis having a window adapted to accommodate a supracondylar Nail or other intramedullary devices required to manage peri-prosthetic fractures in the supracondylar area.

In accordance with the preferred embodiment of the invention, the window is located in the femoral component of the knee prosthesis, particularly in the region between the femoral condyles formed in the femoral component.

The window may be typically rectangular or oval in shape. In the rectangular configuration the window may have the dimensions of a width of 11 mm and a length varying from 23 mm to 34 mm.

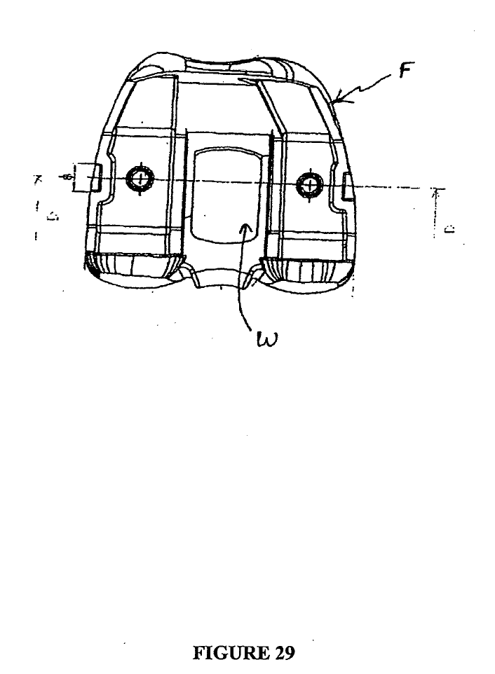

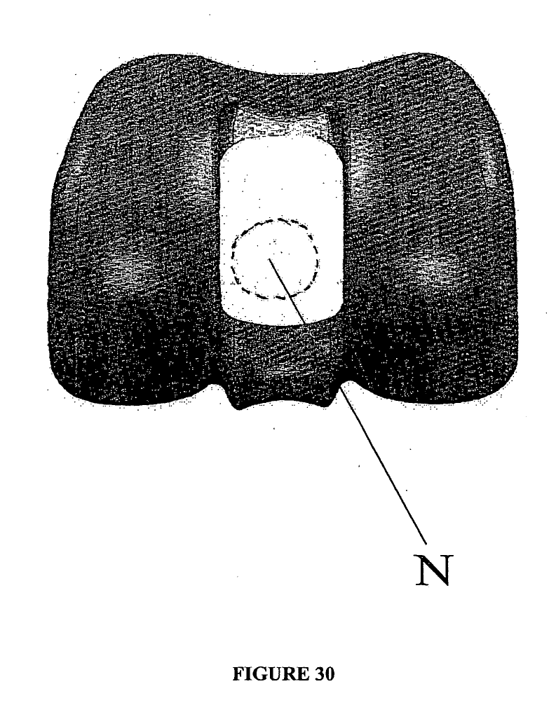

The feature in accordance with this aspect of the invention is illustrated with reference to FIGS. 29 and 30 which are a front view of the femoral component F of the knee prosthesis in accordance with this invention showing the window W through which an intramedullar nail (N) can be inserted through the location shown in the circle in dotted lines.

Fractures can occur in individuals who had undergone knee replacement surgery in the close vicinity of prosthesis (artificial joint) what is termed as PeriProsthetic fractures in the supra condylar femur region. (i.e. above the level of femoral component of the artificial knee joint).

In a normal individual who has not undergone TKR surgery, managing such fractures are easier as various modalities are available which ranges from traction to plaster to plate fixation to Intra medullary nail insertion.

Since there is no prosthesis at the lower end of the femur, managing such fractures is a relatively easy task due to availability of condylar bone portion where the plate can get good hold in bone or one can pass intramedullary nail from the intercondylar region of the femur.

Essentially, surgery for fixing this fracture remains the mainstay of treatment for such fractures for easy mobilization. Two types of surgeries can be performed.

1. Plating:
Plates available for this are:
   DCP—Dynamic Compression Plate
   LCDCP—Low Contact Dynamic Compression Plate
   LCP—Locking Compression Plate

2. Nailing:
Supracondylar Femoral Nails Retrograde Interlocking Nails are inserted through knee joint from intercondylar area for management of the fractures.

Plating requires open surgery i.e. the surgeon needs to open the fracture site. As a result there is much bigger incision and therefore greater blood loss. Further there is also a greater chance of infection. In the open surgical process the haematoma is lost. This haematoma is an important factor in the healing process as it contains osteogenic factors. This loss of the haematoma causes a delay in the healing process. The plate is a load bearing device hence problems of stress shielding are present with the plate. There are also greater chances of osteoporosis taking place under the plate. Therefore, the probability of refracture is high once plate is removed. Further the plate, biomechanically is an extramedullary device and therefore the construct is weak compared to intramedullary nail (medullary nail) and the bending forces are very high resulting into high chances of implant failure. Finally, in the plate fixation and removal procedure, two surgeries are needed: one at the time of insertion of the plate and the other at the time of removal of the plate. The plate has to be removed after about a year and a half to prevent osteoporosis occurring under the plate.

Nailing on the other hand is done by close procedure and therefore minimally opening at the joint level is required. Relatively less time is required for surgery and the operation time is reduced by 20 to 25 minutes as compared to plating. The surgery requires a small incision therefore blood loss is less. Consequently, therefore chances of infection are small as there is small incision. Moreover, the fracture haematoma is preserved and concomitantly, there is faster healing. Since, the Nail is load sharing device rather than a load bearing device there are no problems of stress shielding. Also, as in the case of the plate there is no reason for osteoporosis to develop at the site of nail insertion. Finally, in the case of the nail only a single surgery needs to be performed as the nail can be left in place permanently. A nail typically costs Rs 12000 to 15000 whereas the plate, which is of titanium, can cost between Rs 30,000 to 35,000. Post operatively, the physiotherapy can be more aggressive and therefore recovery is faster.

The aforesaid reasons make nailing a better alternative than plating in fractures in the knee region.

In a replaced knee however, currently, the only option which remains in hand is doing surface fixation i.e. plating surgery. As the solid portion of the existing knee prosthesis in the intercondylar area doesn't allow the nail to be passed through it making nailing impossible in a replaced knee.

Therefore, in a patient who has undergone TKR surgery, there is a femoral prosthesis at the lower end of femur and a limitation arises if a fracture occurs just proximal to the prosthesis what is called as periprosthetic fracture. Use of an intra medullary nail is not possible because minimally invasive procedure cannot be done in replaced knee because it does not allow the nail to be passed from the intercondylar area. The only alternative hitherto is plating.

A particular case will explain the importance of the features of this invention. A 73 year old female suffering from right knee rheumatoid arthritis was admitted for total knee replacement surgery. She underwent TKR surgery. She was doing well postoperatively until she had a fall. Patient started complaining of pain, swelling and deformity around knee joint for which she was again admitted. Patient x-rays were taken and she was diagnosed with periprosthetic fracture in the supra condylar region of the right femur with prosthesis holding well in a distal fragment. Patient was admitted with immobilization in a Thomas splint given to right lower limb. Since the knee prosthesis did not have a window which allowed intra medullary nailing, the plan to fix the fracture using locking compression plate [L.C.P.] was finalized for the patient. She was operated for periprosthetic fracture in the right supra condylar region femur using an L.C.P after making 10 Holes and using 2 Inter Fragmental Screws. During plating the fracture haematoma was washed out. An open surgery was performed under general anesthesia. Postoperatively her limb was immobilized again in a Thomas splint. Patient developed superficial wound infection with wound dehiscence in the scar of second surgery done for L.C. Plating. Daily dressing and antibiotics were administered according to culture sensitivity. Patient's wound improved over a period of 2-3 weeks following which secondary suturing was required to be done. Patient was discharged after complete healing of the wound and was kept under physiotherapy protocol. Patient was allowed to walk with a walker with toe touch weight bearing in initial part of post operative period that was gradually increased to Partial Weight Bearing & Full Weight Bearing at the end of 6 weeks and 12 weeks respectively. Patient's fracture subsequently got united over a period of 3 months when she was allowed full weight bearing. It is not yet time to remove the plate as the plate has to be left in place for around eighteen months at least. But eventually a second operation will have to be performed for removal of the plate.

If the patient had been fitted with a prosthesis in accordance with the window for inserting of an intra medullary nail, she could have been fitted with the nail in a closed reduction surgical operation. The haematoma could have been preserved which would have resulted in better healing. Smaller incision would have prevented the chances of post-operative infections The post operative physiotherapy could have been more aggressive and she could possibly have been out of hospital within one month. The operation would have been less expensive, would have taken less time, and moreover the patient would not have required to come in for a second surgery.

I claim:

1. A knee joint prosthesis comprising (i) a ‘U’ shaped metallic femoral component with one arm longer than the other, the longer arm having an operatively inwardly concave depression within which a patella can be accommodated, the shorter arm being contoured to replicate two femoral condyles of an anatomical knee; a recess provided between said replicated femoral condyles; (ii) a tibial component consisting of a metallic tibial tray element and a tibial plateau element of synthetic polymeric material rigidly shrunk fit in the tibial tray element; said tibial plateau element being hemi oval and having formed therein two laterally spaced apart, kidney shaped trough like meniscal depressions for receiving said replicated femoral condyles; (iii) articulating means on the femoral and the tibial component including a hemi capstan shaped bridge member, having an operative concave surface, on the femoral component and a post, having an operative convex surface complementary to said concave surface, on the tibial component; and (iv) load transfer means comprising triangular web shaped flanges and a stem provided on the operative lower surface of the tibial component.

2. A knee joint prosthesis as claimed in claim 1, in which the femoral component and the tibial tray element is of cobalt chrome alloy and the tibial plateau element is of high density synthetic polymeric material, typically high density polyethylene.

3. A knee joint prosthesis as claimed in claim 1, in which said bridge member is disposed in said shorter arm between said replicated condyles and bridges said recess in the femoral component.

4. A knee joint prosthesis as claimed in claim 1, in which said recess has at least one window through at least a portion of the recess.

5. A knee joint prosthesis as claimed in claim 1, in which the end of the recess distal from the bridge member is concaved.

6. A knee joint prosthesis as claimed in claim 1, in which the longer arm of femoral component terminates in a curved edge.

7. A knee joint prosthesis as claimed in claim 1, in which projecting pins extend operatively inwardly from the inner surface of the U shaped femoral component on either side of the recess.

8. A knee joint prosthesis as claimed in claim 1, in which the operative outer surface of the femoral component is mirror polished and the operative inner surface defines a plurality of recesses for securing the femur to the femoral component in its operative configuration.

9. A knee joint prosthesis as claimed in claim 1, in which the tibial component has formed therein the post extending upwardly from the plateau portion between the mensical depressions.

10. A knee joint prosthesis as claimed in claim 1, in which the post is defined by a truncated pyramid rounded at the top, sectioned at the center along a right angularly disposed concave surface, said post being disposed on the operative posterior side of the tibial plateau between the meniscal depressions.

11. A knee joint prosthesis as claimed in claim 1, in which the post has an operative anterior wall which has a convex smooth surface concaved at the bottom, which contours the bridge member of the femoral component in its operative configuration.

12. A knee joint prosthesis as claimed in claim 1, in which the post has a reinforcing pin provided therein.

13. A knee joint prosthesis as claimed in claim 1, in which the triangular web flanges on either side of the stem define walls joined to the tibial tray, said walls being operatively below and aligned with the short axis of the meniscal depressions in the tibial plateau and extend around the deepest point of the meniscal depressions approximately below the contact area between the contacting bearing surfaces of the replicated condyles and the surfaces of the meniscal depressions in the operative configuration of the prosthesis.

14. A knee joint prosthesis as claimed in claim 1, in which the hemi capstan element and the post element cooperate as a joint in the operative configuration of the prosthesis for transferring load in deep flexion.

15. A knee joint prosthesis as claimed in claim 1, in which the stem is defined by a cylindrical body having a long axis extending operatively at an angle of 7 degrees to a vertical below the tibial tray.

16. A knee joint prosthesis as claimed in claim 1, in which the base of the stem joined to the base of the tibial tray lies approximately below the operative anterior edge of the base of the post whereas the free edge of the stem extends upto the posterior edge of the base of the post.

17. A knee joint prosthesis as claimed in claim 1, in which the end of the stem distal from the tibial tray is provided with threads to accommodates extension rods for additional support for the prosthesis.

18. A knee prosthesis as claimed in claim 1, having a window adapted to accommodate a supracondylar nail or other intramedullary devices required to manage peri-prosthetic fractures in the supracondylar area.

19. A knee prosthesis as claimed in claim 18, wherein the window is rectangular or oval in shape and located in the femoral component of the knee prosthesis in the region between the femoral condyles formed in the femoral component.
